(19) United States
(12) Patent Application Publication
(10) Pub. No.: US 2003/0018391 A1
(43) Pub. Date: Jan. 23, 2003

(54) Total hip joint replacement prosthesis

(76) Inventor: Robert Evans, Chautauqua, NY (US)

Correspondence Address: LATHROP & GAGE LC, 4845 PEARL EAST CIRCLE, SUITE 300, BOULDER, CO, 80301, US

(21) Appl. No.: 10/200,239

(22) Filed: Jul. 22, 2002

Related U.S. Application Data
(60) Provisional application No. 60/307,244, filed on Jul. 23, 2001.

(51) Int. Cl. A61F 2/36

(52) U.S. Cl. 623/23.12

(57) ABSTRACT

To do a total replacement of the hip joint, complete access to the hip joint is achieved through separation of the trochanter, which protrudes from the upper portion of the upper leg bone (the femur) from the rest of the leg, together with dislocation of the hip joint itself. After dislocation the femoral ball is reamed down just enough to accommodate a hemispherical—hollow cap or shell which has approximately the same outside diameter as the patients existing femoral ball prior to reaming. Upon completion of the reaming process the femoral ball remains hemispherical with its diameter and surface reduced by from 4 mm to 5 mm in order to accommodate the metal alloy cap.

### RELATED APPLICATION

Applicant claims priority to U.S. Provisional patent Application Serial No. 60/307,12441 entitled “Total Hip Joint Replacement Prosthesis” filed Jul. 23, 2001.

### FIELD OF THE INVENTION

This invention relates to providing multiple methods of fixation for a resurfacing cap or shell covering the femoral ball of the hip joint versus the present singular cementing or bone ingrowth fixations techniques.

### BACKGROUND OF THE INVENTION

Various partial or complete replacements of the hip joint have been proposed and used since the early 1900's. Most of the procedures or methods involved material problems that led to joint loosening and consequential failure. The widely use total hip replacement which employs the removal of the femoral ball and insertion of a stem into the upper end of the femur to which a small metal ball is affixed, replacing the patient's femoral ball, requires massive bone removal and results in extreme loading of force in a Leveraging action from the initial point of force application i.e. the top of the femoral ball replacement to the lower part of the stem. This force applied by vigorous and mostly younger patients over a few years may cause the stem, which is singularly fixed by either cement or by bone growth into a porous coating to loosen, resulting in subsequent failure, pain and need for extensive expensive revision. Alternate resurfacing of the femoral ball systems have been developed and employed which also employ a singular fixation approach using either cementing or bone ingrowth into a porous surface or surfacing material. With the singular fixation approach there isn't any mechanism to backup, reinforce and absorb the large application downward force and subsequent reactionary force applied though vigorous activity; thus, increasing the possibility of failure.

A principle object of the present invention is to provide a hip joint replacement technique and structure that will require less removal of bone than by procedures and techniques heretofore used. While most importantly maintaining the same femoral head shape as designed by nature so that the transfer of force is not changed together with multiple fixation methods designed to guarantee long term fixation of the femoral cap to the femoral ball. Each method of fixation employs a different approach and absorbs a portion of the applied and reactionary shearing forces that are generated by highly active patients; thus, reducing the possibility of the principle cause of hip joint replacement failure which is loosening.

### SUMMARY OF INVENTION

The present invention involves a concept of multiple total hip joint femoral cap fixation methods requiring less removal of bone that has been previously employed, maintains the same femoral head shape as designed by nature to best accept and most importantly evenly distribute applied and reactionary forces to the femur; thus, lessening the potential for shearing and loosening of the cap or shell. The first method is one of mechanical fixation which employs the engagement of three internal metal non-shear bars into slots cut into the femoral ball deep enough to accommodate the non-shear bars which are from 5-8 mm wide and 4 mm deep. These non-shear bars are an integral part of the hemispherical cap or shell designed to reinforce and strengthen the cap or shell to withstand shear forces many time those which are generated by vigorous activity. Therefore, this method of fixation prevents lateral and longitudinal rotation of the cap or shell while involving only two components which can be sheared or loosened, the cap or shell and bone versus three components employed in the cementing or bone ingrowth fixation technique namely the stem used in total hip replacements or cap/shell used in resurfacing hip replacements the cement or bone ingrowth coating system and the bone itself. This leaves only one direction in which the cap or shell can separate from the femoral ball which is in a due northerly or straight off the femoral ball without rotating. This direction of separation is mechanically prevented through the employment of three metal spring loaded absorption fixators (SLAF's) that screw into the ends of the three internal non-shear bars at their hemispherical ends. The spring loaded absorption fixators (SLAF's) extend downward some 10-15 mm whereby they are approximately separated from the side of the femoral ball some 3 mm. Each spring loaded absorption fixator (SLAF) is then screwed into the femoral ball pulling the tab against the side of the lower femoral ball. The correct load for each spring loaded absorption fixator (SLAF) may be selected from a range of 30# to 130# in order to provide a southerly retention force equal to one third of the patient's body weight on the cap or shell and also absorb a portion of the applied and reactionary forces received by the formal ball similar to normal bone flexation itself. Additionally the screws are prevented from backing out by seating them deep enough within the spring loaded absorption fixator (SLAF) so that a small anti back out metal flat tab can be inserted into a slot running parallel to the head surface of each screw. Once this flat tab is inserted into the slot it drops down into a position lower than the slot itself so that it can not come out without the use of special removal tools. The flat tab sits on top of the head of the screw making it impossible for it to back out. In addition the threads of the bone screws are notched so that once in place bone will grow into the notches providing a back up anti-back out screw fixation method. The lower or southerly end of the spring loaded absorption fixators (SLAF) are also coated with hydroxyapatite porocast or similar system to create fixation via bone ingrowth as well. The second method of preventing a separation or loosening of the cap in a northerly or straight off non-rotational direction is by bone to bone growth through gear tooth shaped teeth which are cast into the bottom surface of the three internal cap bars starting northerly 8 mm from the hemispherical end of each bar and running 15 mm down each bar. Each tooth is 2 mm deep and 2 mm wide and when coated with a hydroxyapatite porocast type system reduces the depth and width to 1 mm providing for substantial purchase through bone growing into the teeth from three directions. Finally a third method of fixation is employed through the coating of the interior of the shell with hydroxyapatite porocast or similar bone ingrowth fixation material which prevents separation or loosening of the cap or shell in all direction.

Other features, advantages and objects of this invention are apparent from a consideration of the following detailed description and associated drawings.

To do a total replacement of the hip joint, complete access to the hip joint is achieved through separation of the trochanter, which protrudes from the upper portion of the upper leg bone (the femur) from the rest of the leg, together with dislocation of the hip joint itself. After dislocation the femoral ball is reamed down just enough to accommodate a hemispherical—hollow cap or shell which has approximately the same outside diameter as the patients existing femoral ball prior to reaming. Upon completion of the reaming process the femoral ball remains hemispherical with its diameter and surface reduced by from 4 mm to 5 mm in order to accommodate the metal alloy cap. Additionally three slots are cut longitudinally equal distance apart in the femoral ball to accommodate three protrubances or non-shear bars within the cap that run perpendicular to the equatorial edge or lip of the cap north/south from its polar orientation a distance of 25 mm to 30 mm. These non-shear bars make it impossible for the cap or shell to separate from the femoral ball through rotation laterally or longitudinally. The cap or shell is also mechanically fixed by three spring loaded absorption fixators (SLAF) which are screwed to the internal longitudinal cap or shell's non-shear bars and subsequently to the femoral ball by 4 mm cancellous screws full thread; thus, securing the cap or shell so that it can not separate from the femoral ball by traveling in a non-rotational direction due north. Additionally the underside of the lower end of the spring loaded absorption fixators (SLAF) are coated with a hydroxyapatite porocast system which provides a second fixation method for the spring loaded absorption fixators (SLAF) through bone growth into a hydroxyapatite porocast type surface. Additionally the internal longitudinal non-shear bars lower surface which faces the bottom of the slots or troughs is designed with a gear tooth configuration which allows the bone to grow more substantially into the teeth and provide a third method of fixing the cap to the femoral ball so that it can not move or separate from the femoral ball in a straight cephalad northerly direction. A fourth method of fixation is achieved through coating the inside surface of the cap between the three non-shear bars with hydroxyapatite porocast or similar system which fixates the cap to the femoral head by bone ingrowth.

### BRIEF DESCRIPTION OF THE DRAWINGS

FIG. 1 shows the upper end of the femur with metal femoral cap and actabular cup;

FIG. 2 shows an arthritic femoral ball;

FIG. 3 shows a cross section of a femoral ball after reaming;

FIG. 4 shows a template device to mark cut location on the femoral head;

FIG. 5 shows a depth gage;

FIG. 6 shows the femoral ball with three slots cut into the ball head;

FIG. 7 shows an internal view of the femoral head cap;

FIG. 8 shows the configuration of the metal spring loaded absorption fixators (SLAF's);

FIG. 9 shows the design of the SLAF;

FIG. 10 shows the femoral cap in place;

FIG. 11 shows the SLAF prior to being screwed down onto the femoral ball;

FIG. 12 shows the SLAF in the compressed state;

FIG. 13 shows the anit-backout locking (ABL) mechanism;

FIG. 14 shows the fixator tabs and ABL;

FIG. 15 shows the SLAF fixator tab prevents the bone screw from backing out;

FIG. 16 shows how the fixator tab may be removed;

FIG. 17 shows the design of the anti-backout 3.5 mm cancellous screw;

FIG. 18 shows an exemplary screw; and

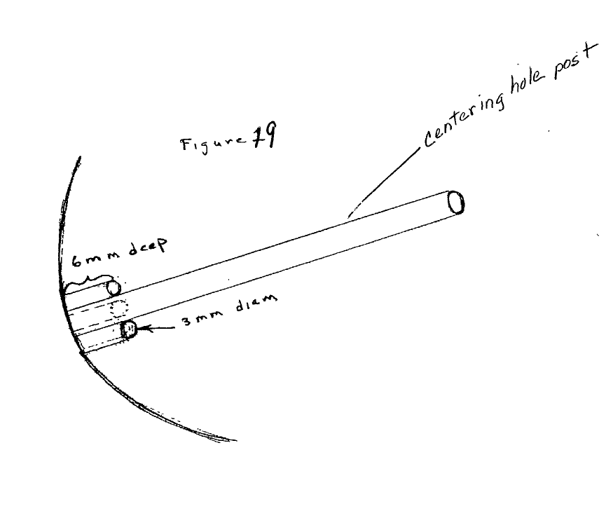

FIGS. 19 & 20 show the centering hole post.

### DETAILED DESCRIPTION OF THE DRAWINGS

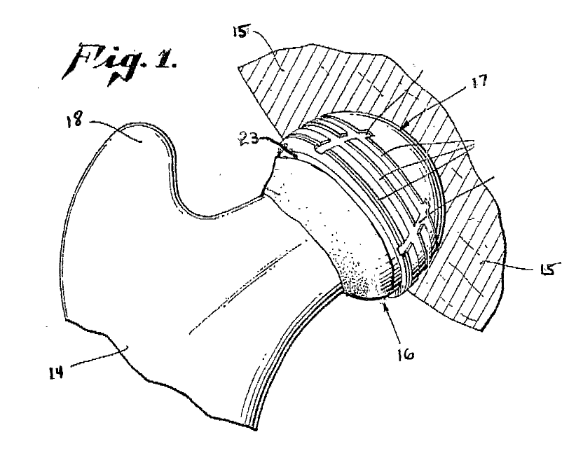

FIG. 1 shows the upper end of the large upper leg bone namely the femur (14) and the pelvis (15) with the metal femoral ball cap or shell (23) and the mating all metal actabular cup (17) shown in position. There is a large protuberance (18) called the trochanter at the upper end of the femur. Full access to the hip joint is obtained by removing this protuberance called the trochanter from the femur (14). Since many of the major muscles controlling the movement of the leg, and which secures the femoral ball (16) into the socked in the hip bone, are attached to the trochanter, separation thereof permits the dislocation of the hip joint fairly easily giving good access to the diseased areas of the arthritic or otherwise diseased hip joint.

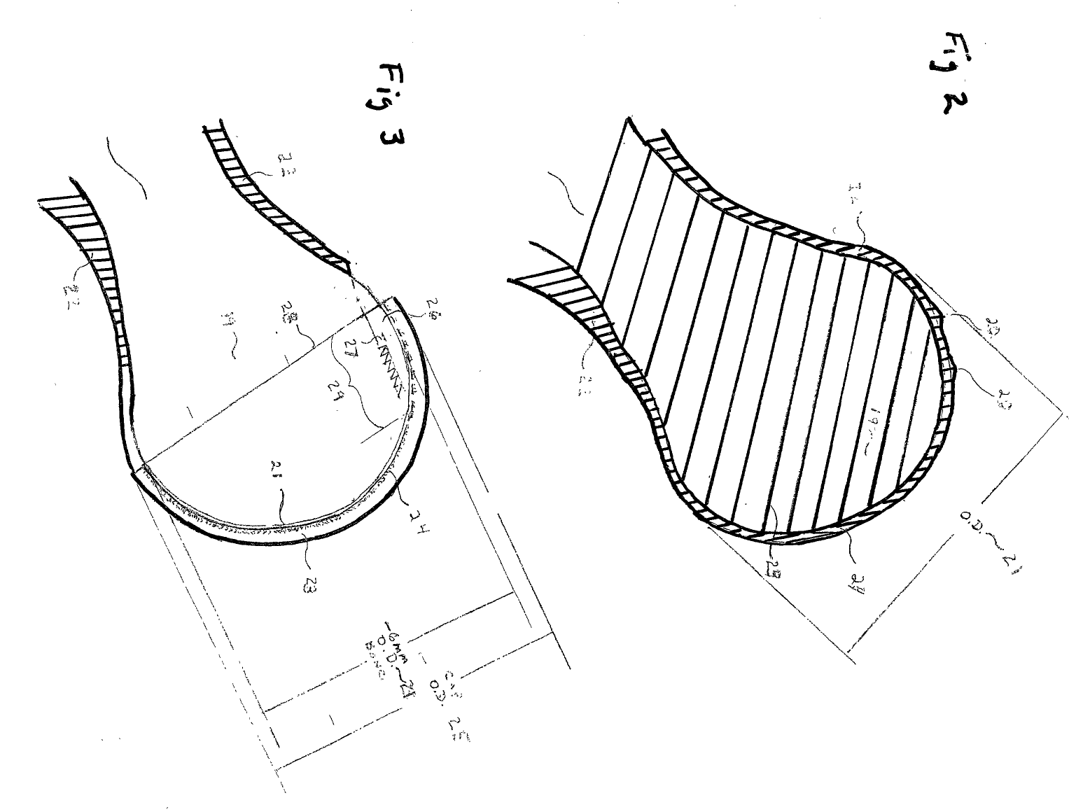

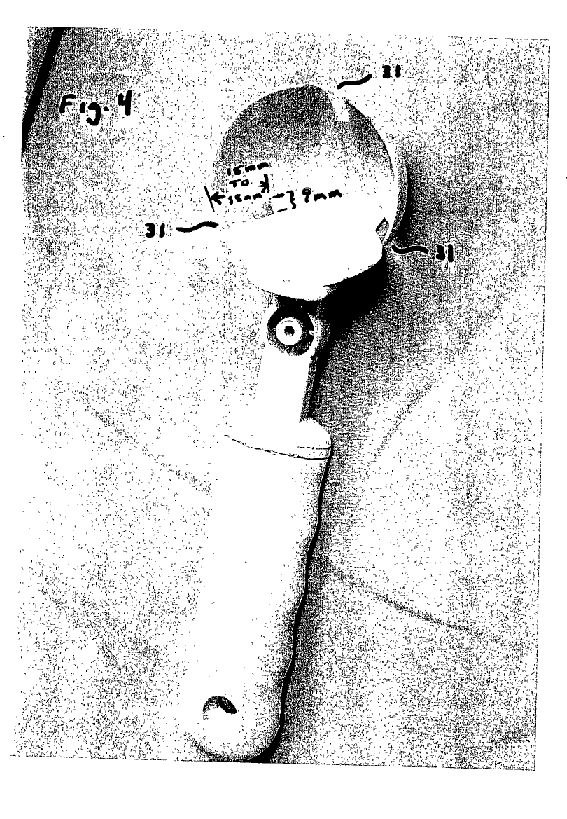

FIG. 2 is a cross sectional view of the femoral ball (19) with some arthritic calcifications (20) and normal femoral ball O.D. (21) prior to reaming off the arthritic calcifications (20) and reduction of the femoral ball (19) to accept the metal cap or shell (22) shown in FIG. 3. The harder outer bone surface (22) is shown in one form of cross sectioning and the strong cacificous inner portion of the bone (23) is shown with a different cross sectioning.

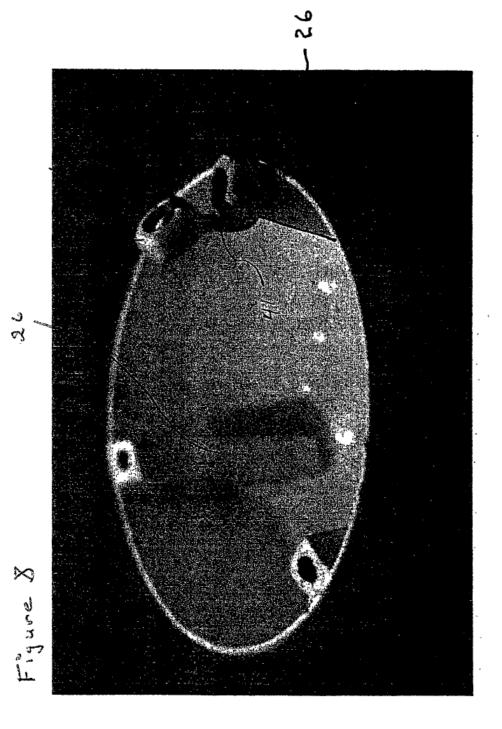

FIG. 3 is a cross sectional view of the femoral ball (19) after uniform down size reaming of the femoral ball by 6 mm (21). This is done without changing the normal shape of the femoral ball. The 6 mm thick cap or shell (23) inner surface is coated with very small protrubances and indentations (24) similar to the hydroxyapatite porocast process which readily accepts bone ingrowth and fixates the cap or shell (23) to the femoral head. Additionally FIG. 3 shows the cap or shell (23) inserted to cover the down sized femoral ball. The cap or shell (23) is selected with an outside diameter (25) that is as close as possible to the original size of the femoral ball (21) prior to reaming as shown in FIG. 1. The harder outer bone surface (22) shown in FIG. 2 is mostly removed from the top and sides of the femoral ball (19) during the reaming and shaping process as shown in FIG. 3. Additionally a side view of one of the three non-shear fixation bars (26) with gear shaped teeth having a depth of 2 mm and a gap of 2 mm wide (27) is shown. These non-shear fixation bars run within the hemispherical cap or shell from the equatorial plane or edge of the cap (28) north 18 mm (29) depending on the size of the cap or shell used. Each non-shear fixation bar extends downward from the inner surface of the hemispherical cap or shell 4 mm (26) with each non-shear fixation bar being 5 to 8 mm in width (See (37) FIG. 7).

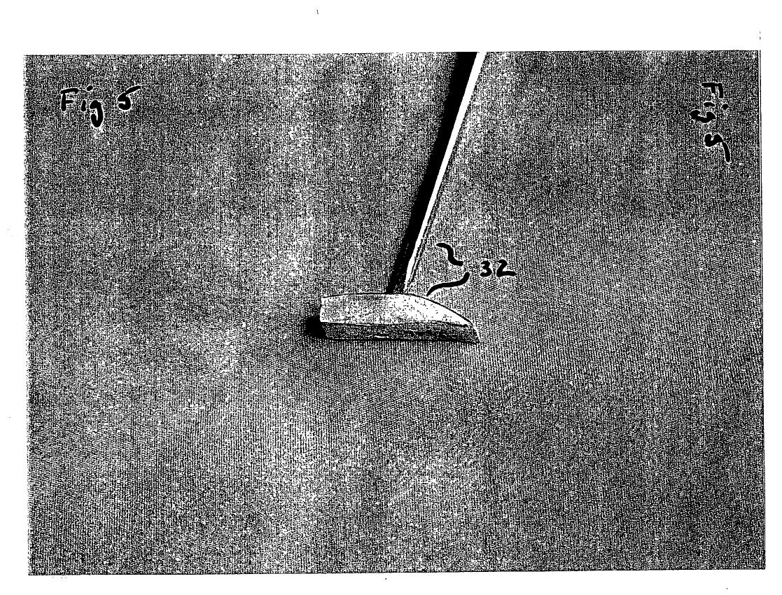

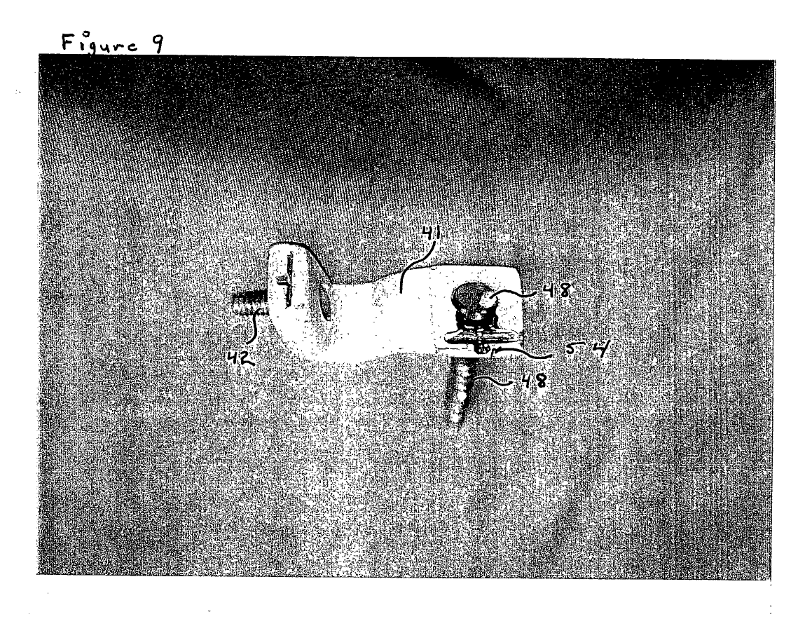

FIG. 4 is an internal view of a template device whose inside diameter is slightly larger than the reamed femoral ball outside diameter (21) shown in FIG. 3. Once the femoral ball (19) has been reamed and downsized the template is placed over the downsized femoral ball and the cut out slots (31) in the template are traced and marked on the downsized femoral ball at the precise location in which the surgeon desires to cut the slots for the non-shear fixation bars (26) shown in FIGS. 3 and 8.

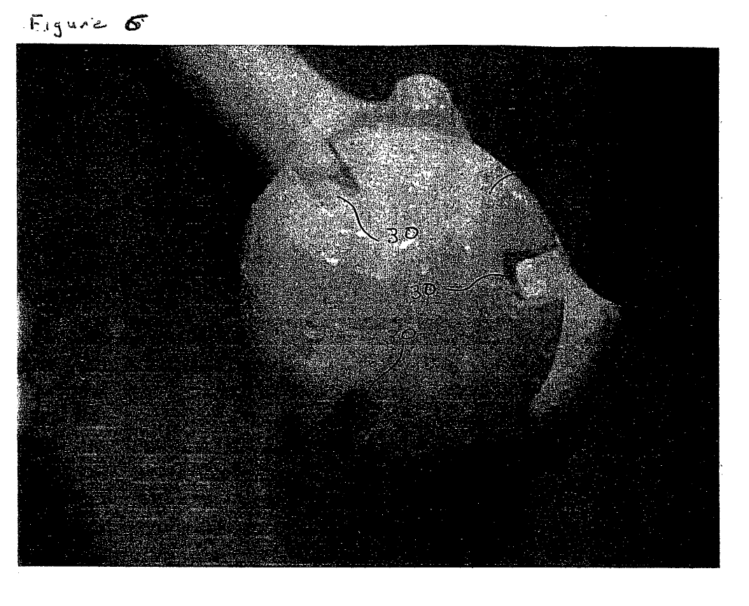

FIG. 5 is a side/bottom view of a depth gauge device (32) developed for the surgeon to use to determine the right depth to cut the traced slots on the downsized femoral ball which will receive the three non-shear fixation bars (26) located on the inner surface of the cap or shell (23).

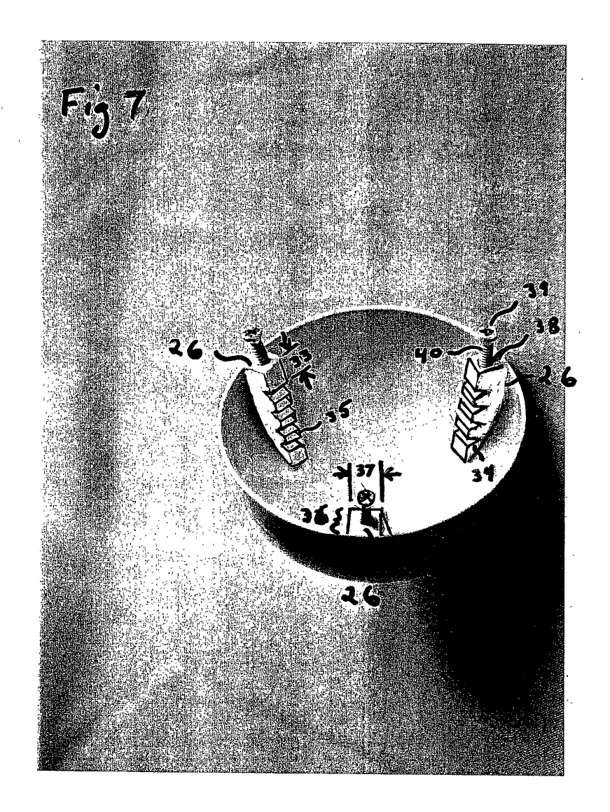

FIG. 6 shows the femoral ball with three slots (30) cut into the femoral ball head in the precise position as determined by the surgeon using the template device shown in FIG. 4 to accept the three non-shear bars (26) located on the inner surface of the cap or shell as shown in FIGS. 3, 7 and 8.

FIG. 7 is an internal view of the cap or shell (16) showing the three non-shear fixation bars (26). The three non-shear fixation bars are 4 mm deep and 7 mm wide and 18 mm long. Each non-shear fixation bar has three teeth cut into the bottom of each bar starting 5 mm back from the front equatorial end of each non-shear fixation bar (33) with each tooth having a flat top 1 mm across with an interior tooth depth of 2 mm (34) and gap width of 2 mm (35). The interior angel or slope of each tooth is 45 degrees. The teeth are coated with the hydroxyapatite porocast type system for bone ingrowth fixation reducing the width and depth of each tooth by 1 mm and increasing the dept of the non-shear fixation bars by 1 mm to a total depth of 5 mm (36). In addition the sides of each non-shear fixation bar are also coated with a hydroxyapatite porocast type system for additional bone ingrowth fixation increasing the width of each non-shear fixation bar by the thickness of the coating and surfacing process approximately 1 mm for a total width ranging from 6-9 mm (37). In addition the equatorial end of each non-shear fixation bar is tapped to receive a 4 mm O.D. diameter machine screw (38) and a head which accepts an allen wrench (39). The thread and material design is specified to withstand a shear force of 1000 pounds (40).

FIG. 8 shows the design configuration of the metal spring loaded absorption fixators (SLAF's) (41) and how they screw into the ends of the three non-shear fixation bars (26).

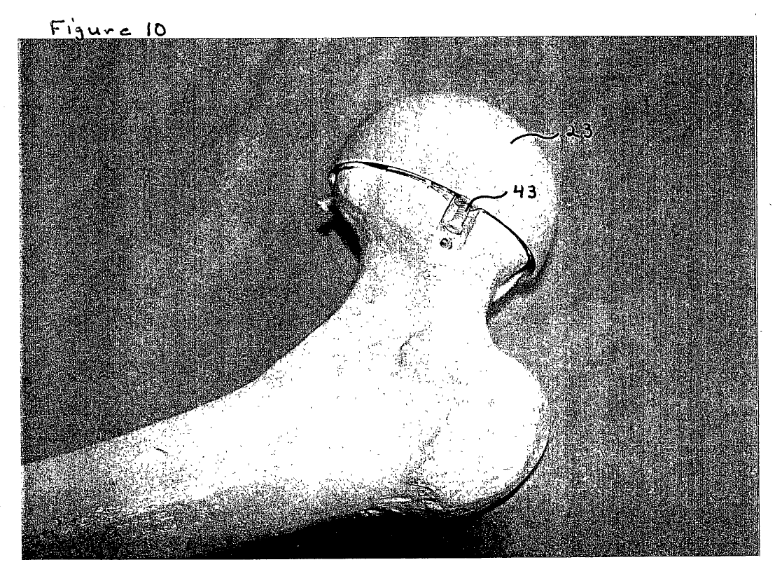

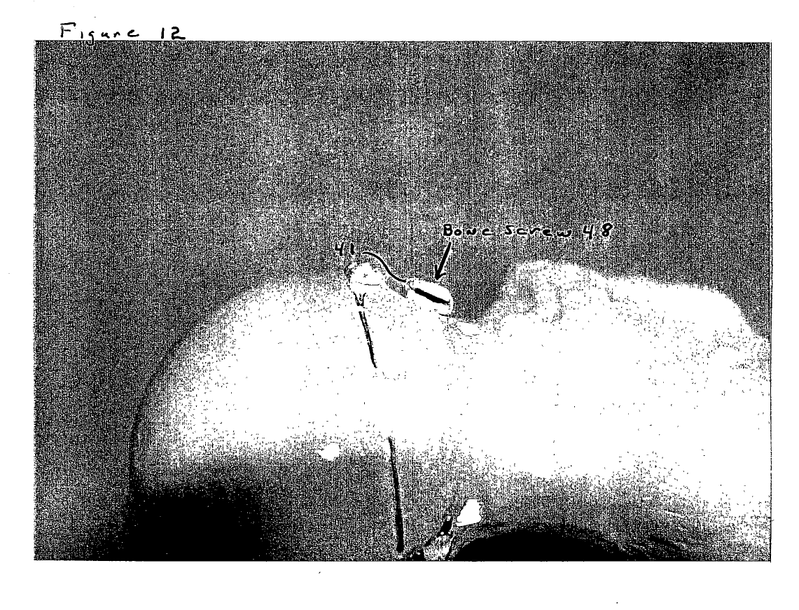

FIG. 9 shows the design of the spring loaded absorption fixators (SLAF's) (41) which utilizes machine screws to attach the SLAF to the end of each non-shear bar (26) as shown in FIGS. 8 and 11.

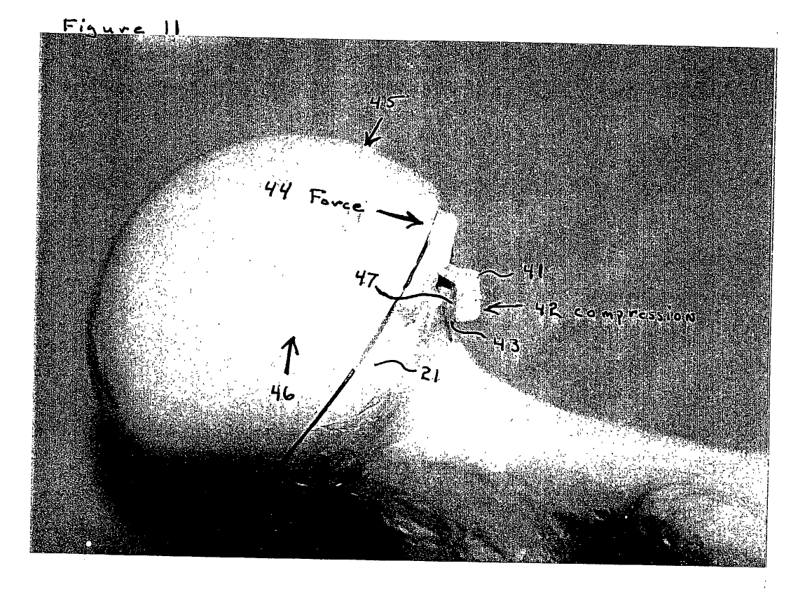

FIG. 10 shows the cap or shell (23) in place capping the down sized femoral ball. The spring loaded absorption fixators (SLAF's) are screwed into the end of each non-shear bar (26) as shown in FIG. 8 with stainless steel machine screws (43).

FIG. 11 shows the metal spring loaded absorption fixators (SLAF's) (41) prior to its being screwed down into the lower portion of the femoral ball (21) just above the neck. The SLAF incorporates a 40 degree bend so that after being screwed into the cap or shell (23) the opposite end is 3 mm off of the femoral ball (21). The compression of the lower end of the spring (42) by 3 mm (43) will deliver a force in direction (44) of from one third to the total weight of the patient depending on which spring load is selected by the surgeon. The correct load for each metal spring loaded absorption fixator (SLAF) may be selected from a range of metal spring loaded absorption fixators (SLAF's) with loads of 30 lbs. to 120 lbs. in 10 lb. Increments in order to provide a total southerly caudal retention force (44) equal to one third of the patient's body weight on the cap or shell and also absorb a portion of the applied and reactionary forces received by the femoral ball similar to the way the amount of flexation of the bone itself does. These metal spring loaded absorption fixators (SLAF's) (41) will thus provide a continuous hold down or fixation force to the cap or shell (23) together with a capability for absorbing and flexing like the bone does with applied downward (45) and reverse reactionary forces (46) making this mechanical method of fixation less susceptible to shearing or breaking loose. Additionally the lower under side area (47) of the metal spring loaded absorption fixators (SLAF's) will be coated with hydroxyapatite porocast type system for additional bone ingrowth fixation.

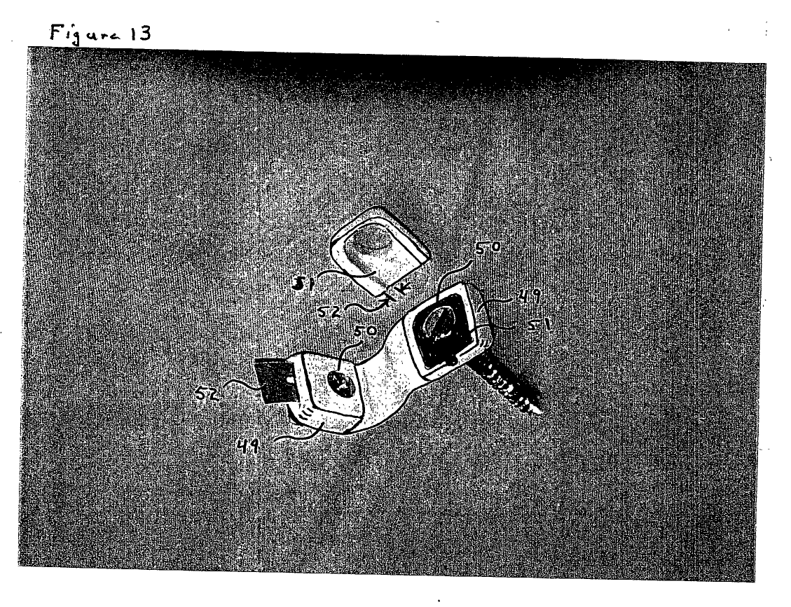

FIG. 12 shows the SLAF (41) in the compressed state after the bone screw (48), as shown in FIG. 9, has been screwed into the lower underside of the femoral ball (21) delivering a southerly fixation force to the shell or cap (23)

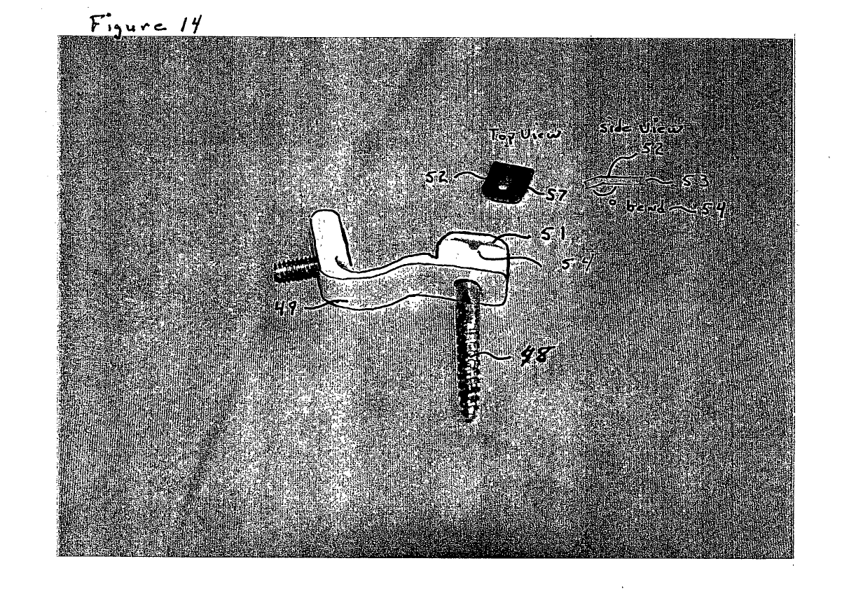

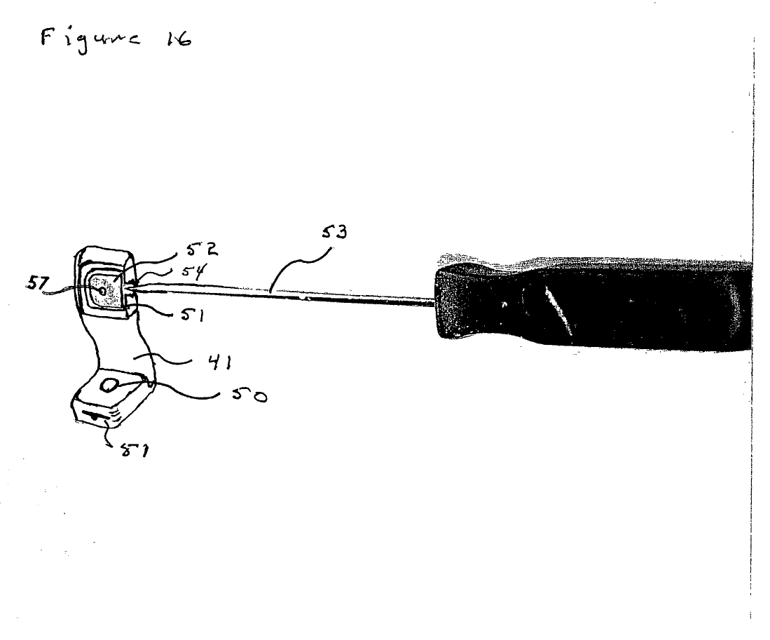

FIG. 13 is a top view of the anit-backout locking (ABL) mechanism (49) which is designed to prevent the screws from loosening or backing out of the bone or non-shear bars. In each end of the ABL a hole (50) is drilled and counter sunk to received the screws. Above the head of each screw is a hollow cavity or slot (51) 1.5 mm thick as also shown in FIG. 15. The right hand end of the ABL which is normally one solid piece but has been split laterally so that the internal cavity or slot (51) may be viewed.

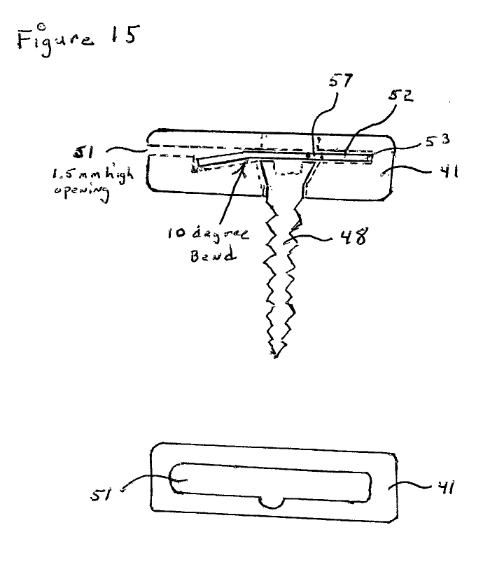

FIG. 14 is a top and side view of the fixator tabs (52) together with a side view of the anti-backout locking (ABL) mechanism (49) showing the slots (51) into which the fixator tabs (52) are inserted to the slot which when in place prevents the screws from backing out. The fixator tabs are 1.4 mm thick (53) and are made of stainless spring steel with a 10 degree downward bend (54) which bends upward when inserted into the slot and once it is all the way into the hollow cavity or slot it snaps back to its original bent shape which prevents it from working out of the slot and locks the screw in place so that it cannot back out. The ABL (49) also is made out of stainless spring steel and is configured so that when attached to the non-shear fixator bars (26 in FIG. 8) by the stainless steel machine screws (38) the other end of the ABL (49) is elevated by 3-4 mm (43 shown in FIG. 11) off of the lower end of the femoral ball (21) so that when screwed down with the bone screw (48) the SLAF (41) delivers a southerly fixation force to the cap or shell (23).

FIG. 15 is an enlarged side and end view of one end of the SLAF (41) showing how the fixator tab (52) after being inserted in the slot (51) prevents the bone screw (48) from backing out and how it snaps down into a position whereby it cannot work itself free of the SLAF (41).

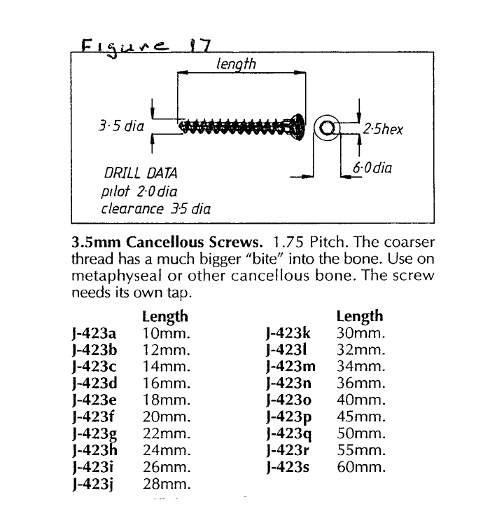

FIG. 16 shows how the fixator tab (52) may be removed by the surgeon if the screws need to be taken out. With a stylist (53) the point of which is inserted in the groove which runs below the fixator tab (56). See FIGS. 9, 14 and 16 (54). Pressure is applied upward to straighten the fixator tab. At the same time a second stylist is inserted though the counter sunk screw hole (50) and into the small hole (57) in the fixator tab pushing the fixator tab (52) out through the slot so the screw may be removed.

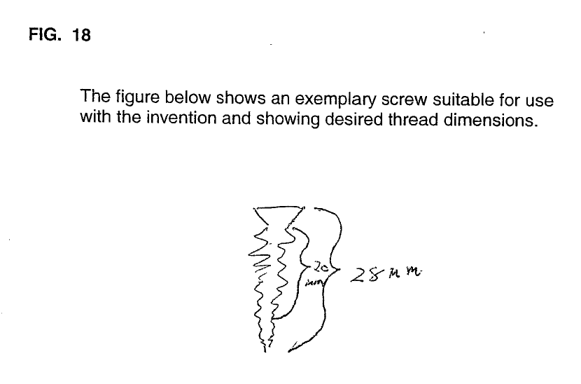

FIG. 17 shows the design of the anti-backout 3.5 mm cancellous screw. The screw has three 0.025 mm deep×0.03 wide notches cut into the threads and running from the head of the screw 70% of its length with each notch being equal distance apart. Once inserted, bone growth can invade these notches; thus, locking the screw in place providing another anit-backout feature.

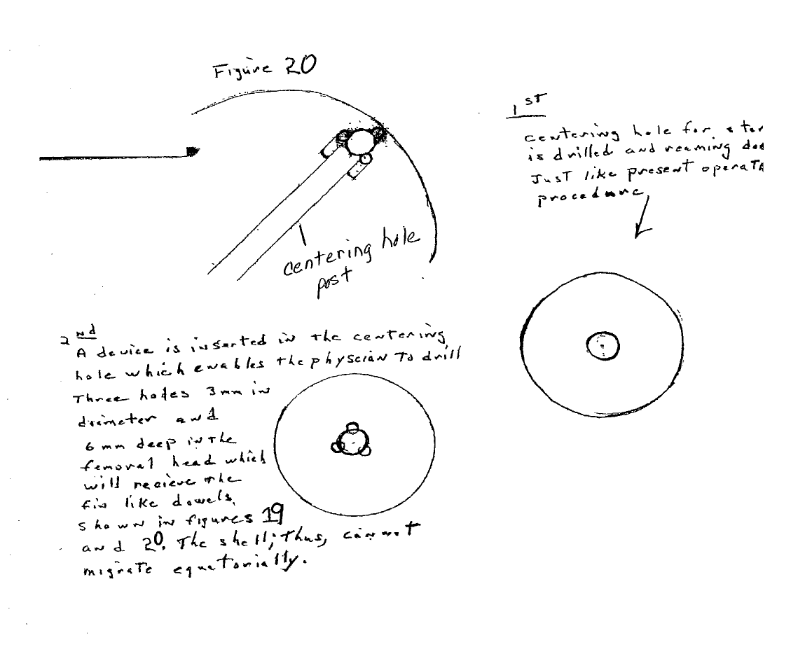

FIGS. 19, 20 shows when a person has enough bone stock, a resurfacing approach may be used. The following shell design (FIGS. 19, 20) have reduced the possibility of the shell rotating longitudinally by reshaping the femoral head into a cylinder. Cement has the function of preventing loosening in the two remaining directions equatorially and polar north. The polar north is not a problem in that anatomically most of the force applied to the femoral head is driving the shell on versus pulling it off. The cement is quite capable of preventing loosening in the polar north direction. This then leaves only the equatorial direction, which is anatomically receiving a larger amount of shear stress. Medical science engineers have identified a slight movement of around 0.08 mm per year; they call this acceptable migration versus slowly loosening. What is happening is the cements' elasticity allows the shell to move due to the application of force. The point when the cements' coefficient of elasticity is reached the cement will fail with significant loosening, requiring a revision. The present posture of the medical science engineers is that the migration or loosening is small enough through the about 5-6 year period in which the present cement has been used, as to not be a problem. Circumstances that can result in failure are:

1. Variance in quality of manufactured cement—Quality control

2. Variance in bone composition from one individual to another which could mean a difference in adhesion

3. Variance in operation technique.

4. Too much or too little cement applied

5. Foreign material getting into the cement

6. Different levels of activity provide significantly higher amounts of force to shell causing varying degrees of migration

Simply put since we know there is migration or loosening and we know there is the potential for accelerated migration or loosening we need a back up technique. With regard to FIGS. 19, 20, migration or loosening of the shell in an equatorial or lateral direction can be stopped by adding three rods or rectangular shaped apertures to the base of the centering hole post that can be inserted into three slots cut into the femoral head. Once seated it will be difficult for the shell to rotate equatorially without shearing the femoral head. Additionally the function of these rods is to provide enough resistance to migration equal to the degree of stress, which is in excess of the cement's ability to hold the shell without migrating. Nonetheless, this back up will allow for additional safety due to some of the variances.

1. Apparatus for resurfacing a femoral ball, comprising (a) a cap having a substantially hemispherical shape of substantially uniform thickness and (b) three non-shear fixation bars for insertion into respective longitudinal slots cut into the femoral ball, wherein the cap remains substantially immovable other than due northerly.

2. Apparatus of claim 1, the non-shear bars comprising three gear shaped teeth to encourage bone growth into 1 mm gaps.

3. Apparatus of claim 1, a symmetrical inner surface of the cap and the sides of the non-shear bars being a hydroxyapatite porocast type system coated to provide and promote bone ingrowth-penetration into the cap facilitating additional fixation for the cap to the femoral head.

4. Apparatus of claim 1, the cap being sized to minimize bone removal from the femoral ball.

5. (Once amended) Apparatus of claim 1, the cap comprising chromium-cobalt-molybdenum alloy that interfaces with a metal cup socket of conventional design maintaining an extremely close clearance tolerance of 0.01 mm, reducing wear and debris which contribute to bone absorption.

6. Apparatus of claim 1, the cap maintaining an original femoral ball size enabling stress forces to dissipate.

7. Apparatus of claim 1, the cap being fixed by mechanical means.

8. Apparatus of claim 7, the mechanical means comprising three metal spring loaded absorption fixators, each fixator being screwed into the cap and femoral ball, preventing loosening of the cap.

9. Apparatus of claim 8, the spring loaded absorption fixators having ability to flex and absorb forces and stress similar to the way a body's bone flexes.

10. Apparatus of claim 8, a lower end of the spring loaded absorption fixators being compressed when screwed into the femoral ball to deliver a constant hold on force to the cap while fixating it to the femoral ball.

11. Apparatus of claim 8, screws employed by the spring loaded absorption fixators being locked via the anti-back out locking mechanism which employs a flat spring steel metal tab inserted into a hollow cavity though a slot which lies just above the head of the screw after it has been screwed to its final position.

12. Apparatus of claim 8, the anit-backout tabs being removable by a surgeon by use of a pointed stylus instrument.

13. Apparatus of claim 1, bone screw threads being uniquely notched in three equidistant spots from the head down 70% of the length of the screw.

14. A method of resurfacing the femoral ball of a hip joint, comprising the steps of:
removing three longitudinal slots of bone in the femoral ball; and attaching a hemispherical cap to the ball, the cap forming three non-shear fixation bars for mating engagement with the slots.

15. The method of claim 14, wherein the step of attaching comprises utilizing three metal spring loaded absorption fixators, and further comprising screwing each of the fixators to the cap and to the femoral ball.

16. A prosthesis for hip joint replacement, comprising:
a hollow cap to mate with three longitudinal slots within a femoral ball of the hip joint, the cap forming non-shear bars for mating engagement with the slots; and three spring-loaded absorption fixators for attaching the cap to the ball.

17. A prosthesis of claim 16, further comprising three screws, each of the screws coupling one of the fixators to the cap and the ball.

18. (Once amended) A prosthesis of claim 16, wherein the fixators comprise a hydroxyapatite porocast coating to facilitate bone growth attachment to the fixators.

19. A prosthesis of claim 16, wherein the cap forms a hollow interior hemisphere surface to accommodate the ball, and further comprising a hydroxyapatite porocast coating to facilitate bone growth attachment to the cap.

20. A method for capping the femoral head with a shell, comprising the steps of: inserting a centering device in a centering hole of the femoral head; drilling three holes about the centering hole, the three holes diameter may vary in size from between about 3 mm to 12 mm and 6 mm in depth; and inserting fin-like dowels which are a part of the centering hole post into the three holes to prevent migration of the shell.
